(19) United States
(12) Patent Application Publication
(10) Pub. No.: US 2020/0305927 A1
(43) Pub. Date: Oct. 1, 2020

(54) BIOPSY SYSTEMS, ULTRASOUND DEVICES, AND METHODS OF USE THEREOF

(71) Applicant: COVIDIEN LP, MANSFIELD, MA (US)

(72) Inventors: Kasey GRIM, Boulder, CO (US); Joe D. SARTOR, Longmont, CO (US)

(21) Appl. No.: 16/806,241

(22) Filed: Mar. 2, 2020

Related U.S. Application Data
(60) Provisional application No. 62/823,177, filed on Mar. 25, 2019.

(51) Int. Cl. A61B 17/34; A61B 8/00; A61B 10/02

(52) U.S. Cl. CPC A61B 17/3403; A61B 8/462; A61B 8/4494; A61B 2017/00924; A61B 10/0233; A61B 2017/3413; A61B 8/445

(57) ABSTRACT

An ultrasound device for guiding a needle includes an elongated handle body and first and second ultrasound transducers. The ultrasound transducers are laterally spaced from one another and angled toward a channel defined therebetween configured for passage of a needle.

### CROSS-REFERENCE TO RELATED APPLICATION

The present application claims the benefit of and priority to U.S. Provisional Patent Application Ser. No. 62/823,177, filed on Mar. 25, 2019, the entire content of which is incorporated herein by reference.

### BACKGROUND

### Technical Field

The present disclosure relates to biopsy sampling and, more particularly, to biopsy systems, ultrasound devices thereof, and methods for navigating a biopsy needle to a target location using the ultrasound device.

### Description of Related Art

To have the best chance of successfully treating cancer, it is critical to diagnose cancer at an early stage. Various methods are used to identify the existence of abnormalities in tissue prior to a patient being symptomatic. For example, women regularly go for prophylactic mammograms to determine whether there are any early stage tumors developing in their breast tissue. Although mammography is effective at identifying whether a tumor is present, mammography is not capable of differentiating between benign and malignant tumors. Accordingly, upon identifying an abnormality in the tissue, the status of the abnormality needs to be determined using an additional diagnostic technique.

One method to verify whether a tissue is cancerous is to obtain a tissue sample for histological examination through a biopsy of the tissue (e.g., breast tissue) near the lesion. There are a number of devices and methods for performing a biopsy. In some instances, a tumor may be identified using manual palpation of the breast tissue and then a biopsy needle may be positioned over the identified tumor to take a sample of tissue. Another method involves holding an ultrasound probe in one hand while holding the biopsy needle with a second hand and guiding the biopsy needle along the image plane of the ultrasound probe.

### SUMMARY

Provided in accordance with the present disclosure is an ultrasound device for guiding a needle and includes an elongated handle body having a first end portion and a second end portion, a display coupled to the first end portion of the handle body, and at least one ultrasound transducer coupled to the second end portion of the handle body. The ultrasound transducer defines a channel configured for passage of a needle. The ultrasound transducer is disposed in operable communication with the display to enable display of an ultrasound image generated by the ultrasound transducer on the display. The ultrasound transducer is configured to direct ultrasound waves inwardly toward the channel.

In aspects, the ultrasound transducer includes first and second ultrasound transducers defining the channel therebetween.

In aspects, the first ultrasound transducer may have a distally-oriented surface defining a first plane, and the second ultrasound transducer may have a distally-oriented surface defining a second plane. The first and second planes may be disposed relative to one another at an angle of between 80 degrees and 170 degrees.

In aspects, the angle between the first and second planes may be between 140 degrees and 165 degrees.

In aspects, the ultrasound device may further include a coupling interface disposed within a cavity cooperatively defined by the first and second ultrasound transducers.

In aspects, the coupling interface may be fabricated from an acoustically-transparent material.

In aspects, the coupling interface may have a peak and define a channel through the peak configured for passage of a needle.

In aspects, the channel of the coupling interface may be aligned with the channel defined between the first and second ultrasound transducers.

In aspects, the coupling interface may have a planar, base surface and each of the first and second ultrasound transducers may have a planar, base surface disposed at an acute angle relative to the base surface of the coupling interface.

In aspects, each of the first and second ultrasound transducers may be disposed on opposite sides of the channel and face a longitudinal axis defined by the channel.

In aspects, the handle body may extend at an angle away from the first and second transducers, such that the display is out of alignment with the first and second transducers.

In aspects, the handle body may have an undulating shape.

In aspects, the ultrasound device may further include a disposable cannula configured for removable receipt in the channel. The cannula may define a longitudinally-extending passageway configured for passage of a needle.

In aspects, the ultrasound device may further include a disposable cap configured to be detachably coupled to the second end portion of the handle body for covering the ultrasound transducer. The cannula may have a distal end portion configured to be detachably coupled to the cap.

In aspects, the display may be slidable relative to the handle body.

In accordance with further aspects of the present disclosure, an ultrasound device for guiding a needle is provided and includes an elongated handle body having a first end portion and a second end portion, a housing coupled to the second end portion of the handle body, and first and second ultrasound transducers disposed within the housing. The housing defines a channel configured for receipt of a needle. The ultrasound transducers are disposed on opposite sides of the channel and angled toward a longitudinal axis defined by the channel.

In aspects, the first ultrasound transducer may have a distally-oriented surface defining a first plane, and the second ultrasound transducer may have a distally-oriented surface defining a second plane. The first and second planes may be disposed relative to one another at an angle of between 80 degrees and 170 degrees.

In aspects, the ultrasound device may further include a coupling interface disposed within a cavity cooperatively defined by the first and second ultrasound transducers. The coupling interface may be fabricated from an acoustically-transparent material.

In aspects, the coupling interface may define a channel therethrough configured for passage of a needle. The channel of the coupling interface may be coaxial with the channel of the housing.

In aspects, the coupling interface may have a planar, base surface and each of the first and second ultrasound transducers may have a planar, base surface disposed at an acute angle relative to the base surface of the coupling interface.

As used herein, the term “distal” refers to the portion that is being described which is further from a user, while the term “proximal” refers to the portion that is being described which is closer to a user. Further, to the extent consistent, any of the aspects and features detailed herein may be used in conjunction with any or all of the other aspects and features detailed herein.

As used herein, the terms parallel and perpendicular are understood to include relative configurations that are substantially parallel and substantially perpendicular up to about + or −10 degrees from true parallel and true perpendicular.

### BRIEF DESCRIPTION OF THE DRAWINGS

Various aspects and features of the present disclosure are described hereinbelow with references to the drawings, wherein:

FIG. 1 is a perspective view illustrating an ultrasound device provided in accordance with the present disclosure configured for guiding a needle to a target location;

FIG. 2A is a perspective view illustrating a tissue biopsy system including the ultrasound device of FIG. 1 and a needle inserted into the ultrasound device;

FIG. 2B is an enlarged perspective view of a distal portion of the tissue biopsy system shown in FIG. 2A;

FIG. 3 is a perspective, partially-transparent view illustrating a support block and a coupling wedge of the ultrasound device of FIG. 1;

FIG. 4 is a perspective view illustrating first and second ultrasound transducers of the ultrasound device of FIG. 1;

FIG. 5 is a side cross-sectional view illustrating the ultrasound device of FIG. 1;

FIG. 6 is a schematic illustration of the first and second ultrasound transducers interfacing with the coupling wedge;

FIG. 7 is an enlarged perspective view illustrating a needle guide assembly coupled to a probe head of the ultrasound device of FIG. 1;

FIG. 8 is a perspective view illustrating the needle guide assembly of FIG. 7; and

FIG. 9 is a perspective, partially-transparent view illustrating a display separated from the handle body of the ultrasound device of FIG. 1.

### DETAILED DESCRIPTION

Needle-guiding ultrasound devices, biopsy systems, and methods for obtaining a tissue sample using the biopsy systems are provided in accordance with the present disclosure and described in detailed below. In one embodiment, the ultrasound device includes an elongated handle body, a display supported by a first end of the handle body, and a pair of first and second ultrasound transducers supported in a probe head coupled to the second end portion of the handle body. The ultrasound transducers are laterally spaced from one another to allow for the passage of a biopsy needle therebetween. The ultrasound transducers are set at an angle relative to one another to each face a path along which the biopsy needle travels during use. Each of the two discrete ultrasound transducers transmits and receives independently, enabling the generation of two separate 2D ultrasound images that are then combined to create a singular, integrated 2D image displayed on the display.

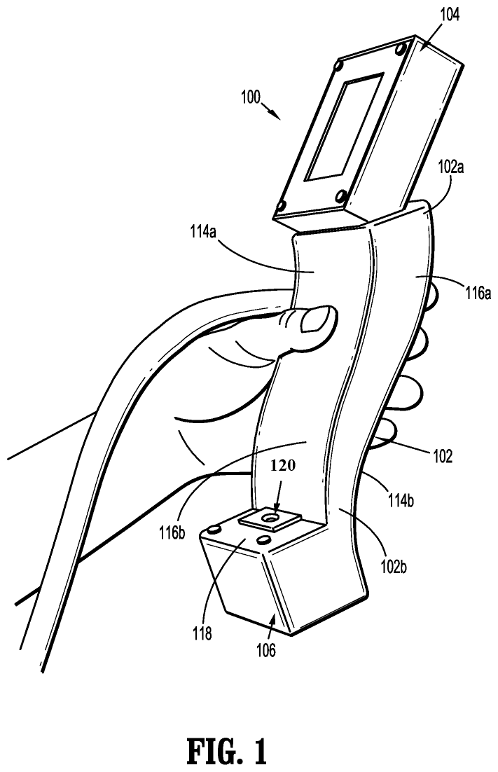

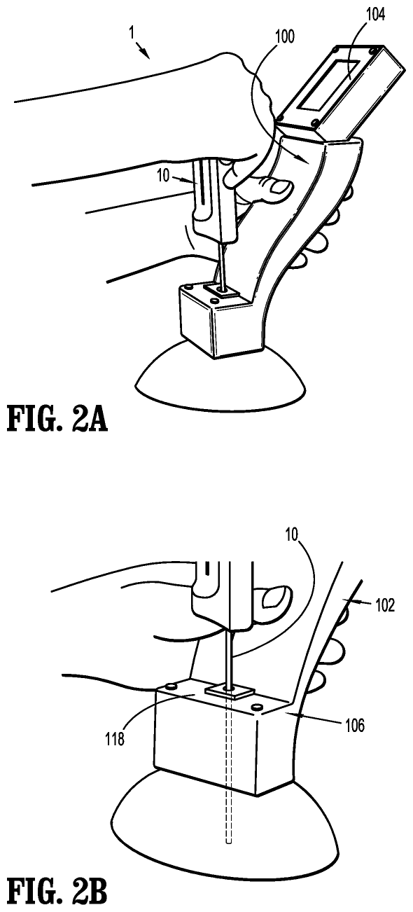

With reference to FIGS. 1, 2A, and 2B, an ultrasound device 100 of a tissue biopsy system 1 is provided in accordance with the present disclosure for guiding and imaging a biopsy needle 10 to obtain a tissue sample from a target tissue, for example, a lesion. The ultrasound device 100 generally includes an elongated handle body 102, a display 104, a probe head 106, and first and second ultrasound transducers 108, 110 (FIG. 4) supported in the probe head 106.

The ultrasound transducers 108, 110 are configured to send ultrasound waves toward a selected tissue site, whereby the tissue site, based upon its physical characteristics, reflects ultrasound waves back to the ultrasound transducers 108, 110, which detect the reflected ultrasound waves and send corresponding signals to a central processing unit (not shown) of the ultrasound device 100. The central processing unit generates an image of the biopsy needle 10 and tissue site based upon the signals received from each of the ultrasound transducers 108, 110 and combines the two separate 2D ultrasound images into a 2D image that is output for display on the display 104.

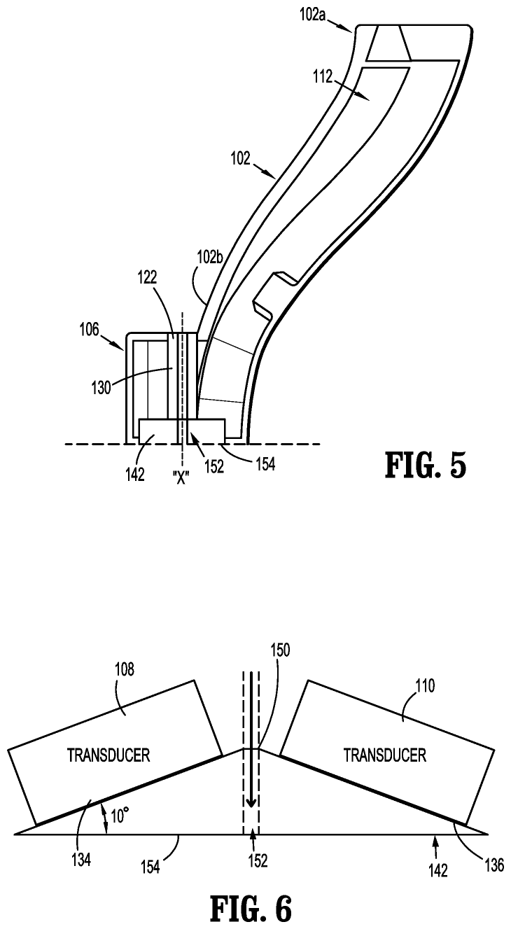

The handle body 102 is fabricated from plastic, such as, for example, PEEK, and has a first end portion 102a supporting the display 104 and a second end portion 102b supporting the probe head 106. Other suitable materials from which the handle body 102 is formed are contemplated. The handle body 102 defines a hollow interior 112 (FIG. 5) through which cables, wires, or the like extend to interconnect the central processing unit with display 104 and the first and second ultrasound transducers 108, 110. The handle body 102 may have a generally undulating shape defining a first concave-shaped trough 114a on a first side of the handle body 102, and a first convex-shaped peak 116a on a second, opposite side of the handle body 102. The first trough and peak 114a, 116a overlap one another, whereby the first trough 114a is configured for receipt of a thumb of a hand of a user, and the first peak 116a is configured for grasping by an index finger and middle finger of a hand of a user. The elongate body 102 has a second, convex-shaped peak 116b disposed on the first side of the handle body 102, and a second, concave-shaped trough 114b disposed on the second side of the handle body 102. The second trough and peak 114b, 116b overlap with one another, whereupon the second trough 114bis configured for receipt of a ring finger and little finger of a hand of a user. In aspects, the handle body 102 may assume any suitable shape, such as, for example, linear or arcuate. Likewise, different grasping configurations are also contemplated. The handle body 102 may house a memory (e.g., an EEPROM—not shown), together with or separate from the central processing unit, for storing a variety of information regarding the ultrasound device 100.

The probe head 106 houses the ultrasound transducers 108, 110 therein and is configured to guide the biopsy needle 10 therethrough. The probe head 106 may have a block-shape and may be monolithically formed with the second end portion 102b of the handle body 102. In other aspects, the probe head 106 may assume any suitable shape and/or may be otherwise connected to the second end portion 102b of the handle body 102. The handle body 102 extends at an angle away from the probe head 106, such that the display 104 is out of alignment with the probe head 106 and its components (e.g., the first and second transducers 108, 110). In this way, when a clinician is operating the ultrasound device 100, the hand of the clinician is out of the way of the probe head 106, and therefore the pathway through which the biopsy needle 10 travels. The probe head 106 has an upper plate 118 defining an entry opening 120 therein for receipt of a biopsy needle, such as, for example, the biopsy needle 10 (FIGS. 2A and 2B).

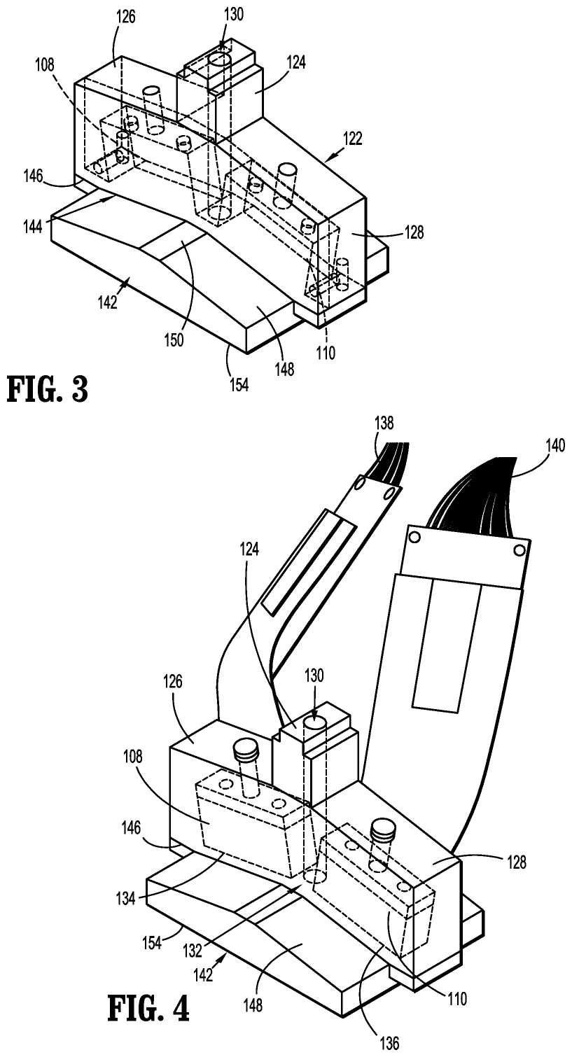

With reference to FIGS. 3-6, the probe head 106 has a support block 122 disposed therein that may be molded or otherwise formed. The support block 122 includes a spine 124 and first and second side portions 126, 128 extending from opposite sides of the spine 124. The spine 124 defines a longitudinally-extending channel 130 therethrough in communication with the entry opening 120 in the upper plate 118 (FIG. 1) of the probe head 106. The channel 130 of the support block 122 is configured for receipt of the biopsy needle 10.

The ultrasound transducers 108, 110 may be molded into pockets formed in the respective first and second side portions 126, 128 of the support block 122. The ultrasound transducers 108, 110 are set within the support block 122 at an angle relative to one another and laterally spaced from one another to define a channel 132 therebetween. As such, the channel 130 of the support block 122 extends between the first and second ultrasound transducers 108, 110, whereby the angled configuration of the first and second ultrasound transducers 108, 110 orients the transducers 108, 110 toward a longitudinal axis “X” defined by the channel 130.

More specifically, each of the first and second ultrasound transducers 108, 110 has a distally-oriented, planar base surface 134, 136 that transmits ultrasound waves therefrom. The base surface 134 of the first transducer 108 defines a plane, and the base surface 136 of the second transducer 110 defines a plane that intersects the plane of the first transducer 108 at an angle of between about 80 degrees and about 170 degrees (wherein “about” takes into account generally accepted tolerances, e.g., material, manufacturing, environmental, measurement, and use tolerances). In embodiments, the angle between the base surfaces 134, 136 may be between about 140 degrees and about 170 degrees, and in some embodiments, about 160 degrees. This angle may be adjusted to optimize the imaging field along the centerline at some point ranging from the tissue contact with the system to some required depth. The angle of the transducers 108, 110 affects the depth of penetration of the ultrasound waves into tissue as well as the allowable distance between the transducers 108, 110. Acute angles are more useful to image shallow tissue and provide better imaging of a biopsy needle that is more perpendicular to the ultrasound path. Each of the transducers 108, 110 has a cable 138, 140, such as, for example, a flex circuit extending therefrom that electrically connects to the central processing unit for transmitting electrical signals (e.g., electrical signals representing the reflected ultrasound waves sensed by transducers 108, 110) thereto for processing and output to the display 104.

In aspects, instead of having two discrete ultrasound transducers 108, 110, the support block 122 may house one ultrasound transducer that has two, angled surfaces for directing ultrasound waves inwardly toward a needle path. The single ultrasound transducer may define the channel 132 therethrough configured for passage of the needle. In other aspects, the ultrasound transducers 108, 110 may act as a single sensor configured to form one image rather than two images that are merged into a single image.

The ultrasound device 100 includes a coupling interface, such as, for example, a wedge 142 disposed within a cavity 144 defined by a distally-oriented, bottom surface 146 of the support block 122. Due to the first and second transducers 108, 110 being angled relative to one another and towards the longitudinal axis “X” of the channel 130 of the support block 122, the cavity 144 may assume a substantially triangular configuration. The coupling wedge 142 is fabricated from an acoustically-transparent material, such as, for example, PEEK, silicone, polyurethane, etc., and has an upper surface 148 that complementarily engages the bottom surface 146 of the support block 122. The coupling wedge 142 closes a gap between the bottom surface 134, 136 of the transducers 108, 110 and a skin surface during use, thereby facilitating the transmission of ultrasound waves from the transducers 108, 110 into tissue.

The upper surface 148 of the coupling wedge 142 has a peak 150 abutting a distal end of the spine 124 of the support block 122. The coupling wedge 142 defines a channel 152 through the peak 150 configured for passage of a biopsy needle. The channel 152 of the coupling wedge 142 is coaxial with the channel 130 of the support block 122 to allow for the passage of a biopsy needle through the support block 122, the coupling wedge 142, and into tissue.

The coupling wedge 142 has a base surface 154 that is planar or otherwise configured and is oriented toward tissue. The base surface 134, 136 of each of the first and second ultrasound transducers 108, 110 is disposed at an acute angle relative to the base surface 154 of the coupling wedge 142. In aspects, the acute angle may be between about 5 degrees and about 20 degrees, and in some embodiments, about 10 degrees. The coupling wedge 142 may be fabricated and subsequently affixed to the transducers 108, 110 or may be molded around the transducers 108, 110.

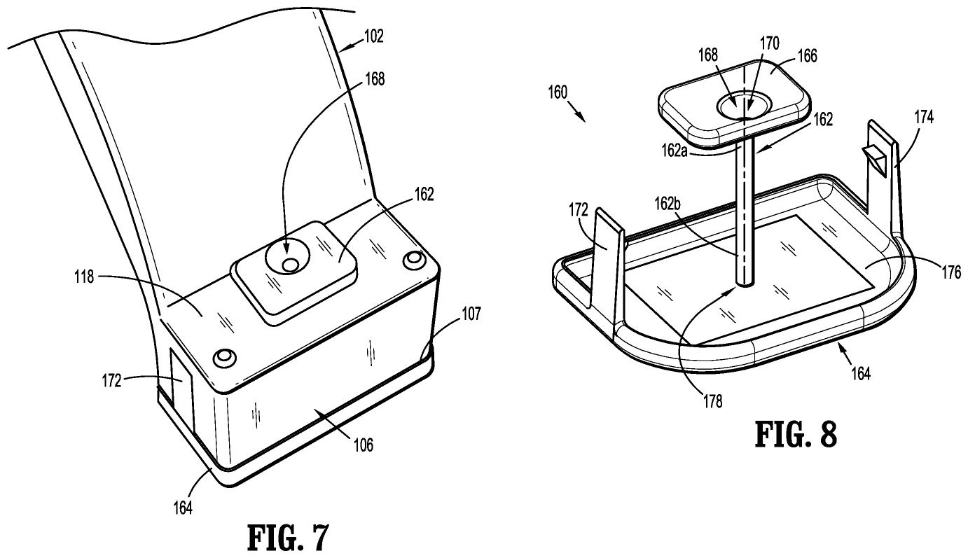

With reference to FIGS. 7 and 8, the biopsy system 1 may include a disposable needle guide assembly 160 configured to be detachably coupled to the probe head 106. The disposable needle guide assembly 160 includes a cannula 162 and a cap 164. The cannula 162 is configured for removable receipt in the channel 130 (FIGS. 3-5) defined through the support block 122 and the channel 152 defined through the coupling wedge 142. The cannula 162 has a proximal end portion 162a supporting an upper plate 166, and a distal end portion 162b. The upper plate 166 of the cannula 162 defines an enlarged entry opening 168 to facilitate positioning the biopsy needle 10 into a longitudinally-extending passageway 170 defined by the cannula 162. The upper plate 166 of the cannula 162 is configured to be positioned on the upper plate 118 of the probe head 106.

The cap 164 of the disposable needle guide assembly 160 is configured to be detachably coupled to a distal end 107 of the probe head 106 for enclosing the first and second ultrasound transducers 108, 110. The cap 164 permits ultrasound propagation therethrough while preventing the coupling wedge 142 from directly contacting a patient. The cap 164 may have a pair of tabs 172, 174 extending proximally therefrom for detachable, snap-fit engagement with a corresponding pair of recesses (not explicitly shown) formed in opposite lateral sides of the probe head 106. The cap 164 has an underside 176 that defines an opening 178 therein at a central location thereof. The opening 178 is configured for receipt of the distal end portion 162b of the cannula 162, such that upon assembling the disposable needle guide assembly 160 to the probe head 106, the cannula 162 is detachably coupled to the cap 164 while allowing for one continuous passageway for a biopsy needle to travel through.

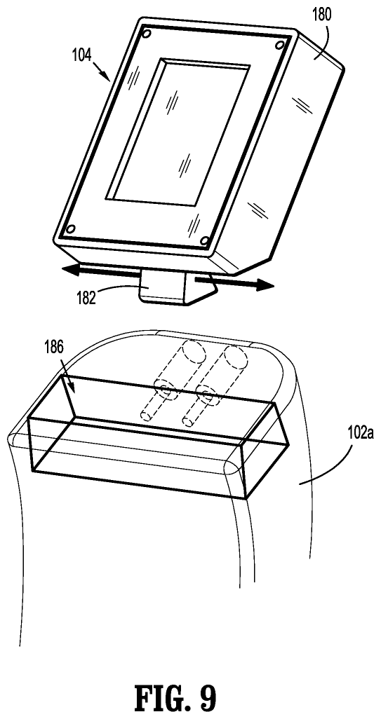

With reference to FIG. 9, the display 104 may be slidable relative to the handle body 102. In particular, the display 104 may have a housing 180 including a flange 182 extending therefrom. The flange 182 is slidably received in a track 186 defined across the first end portion 102a of the handle body 102. In this way, the display 104 may be moved to a variety of lateral positions relative to the handle body 102 in instances where a clinician's hand may otherwise be blocking a view of the display 104, for example, to accommodate both left-handed and right-handed use.

In some aspects, the biopsy system 1 may implement Doppler imaging from one or both ultrasound transducers 108, 110 as needed to avoid vascular structures in the needle path.

Referring generally to FIGS. 1-9, one use of the ultrasound device 100 for extracting tissue samples from a lesion, e.g., a tumor, will now be described. With the disposable needle guide assembly 160 coupled to the probe head 106, the ultrasound device 100 is positioned such that the cap 164 is placed in abutting engagement with an outer surface of tissue (e.g., breast tissue). The ultrasound transducers 108, 110 are activated to emit ultrasound waves toward a lesion within the breast tissue. The transducers 108, 110 then receive the reflected ultrasound waves and transmit corresponding signals to the central processing unit (not shown) which generates an image of the lesion and surrounding breast tissue. The biopsy needle 10 is positioned into the entry opening 168 of the cannula 162 and passed through the passageway 170 of the cannula 170 and between the transducers 108, 110. Since the transducers 108, 110 are both angled toward the needle path, the ultrasound waves emitted by the transducers 108, 110 reflect off of the biopsy needle 10 as it passes therebetween. The two separate 2D ultrasound images generated based upon the signals provided by the ultrasound transducers 108, 110 are combined to create a singular, integrated 2D image using an algorithm (according to any suitable image generation and processing techniques) stored in the memory of the central processing unit. The integrated 2D image is displayed on the display 104, enabling both visualization of the breast tissue and the needle 10, thus assisting the clinician in aligning the needle 10 with the lesion. If it is determined that the needle 10 is out of alignment with the lesion, the ultrasound device 100 may be moved relative to the lesion, in turn moving the needle 10, until the needle 10 is at the appropriate location.

In aspects, the integrated 2D image may visually indicate the optimal tissue area to target based on image characteristics, such as, for example, density or texture.

Upon the display 104 showing the needle 10 aligned with the lesion, the needle 10 may be moved manually in the distal direction to penetrate the lesion. As the needle 10 penetrates the lesion, a tissue sample of the lesion enters the needle 10.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An ultrasound device for guiding a biopsy needle, the ultrasound device comprising:
an elongated handle body having a first end portion and a second end portion;
a display coupled to the first end portion of the handle body; and
at least one ultrasound transducer coupled to the second end portion of the handle body and defining a channel configured for passage of a needle, the at least one ultrasound transducer disposed in operable communication with the display to enable display of an ultrasound image generated by the at least one ultrasound transducer on the display, wherein the at least one ultrasound transducer is configured to direct ultrasound waves inwardly toward the channel.

2. The ultrasound device according to claim 1, wherein the at least one ultrasound transducer includes first and second ultrasound transducers defining the channel therebetween.

3. The ultrasound device according to claim 2, wherein the first ultrasound transducer has a distally-oriented surface defining a first plane, and the second ultrasound transducer has a distally-oriented surface defining a second plane, the first and second planes disposed relative to one another at an angle of between 80 degrees and 170 degrees.

4. The ultrasound device according to claim 3, wherein the angle between the first and second planes is between 140 degrees and 165 degrees.

5. The ultrasound device according to claim 2, further comprising a coupling interface disposed within a cavity cooperatively defined by the first and second ultrasound transducers.

6. The ultrasound device according to claim 5, wherein the coupling interface is fabricated from an acoustically-transparent material.

7. The ultrasound device according to claim 5, wherein the coupling interface has a peak and defines a channel through the peak configured for passage of a needle.

8. The ultrasound device according to claim 7, wherein the channel of the coupling interface is aligned with the channel defined between the first and second ultrasound transducers.

9. The ultrasound device according to claim 5, wherein the coupling interface has a planar, base surface and each of the first and second ultrasound transducers has a planar, base surface disposed at an acute angle relative to the base surface of the coupling interface.

10. The ultrasound device according to claim 7, wherein each of the first and second ultrasound transducers is disposed on opposite sides of the channel of the coupling interface and faces a longitudinal axis defined by the channel of the coupling interface.

11. The ultrasound device according to claim 2, wherein the handle body extends at an angle away from the first and second ultrasound transducers, such that the display is out of alignment with the first and second ultrasound transducers.

12. The ultrasound device according to claim 1, wherein the handle body has an undulating shape.

13. The ultrasound device according to claim 1, further comprising a disposable cannula configured for removable receipt in the channel, the cannula defining a longitudinally-extending passageway configured for passage of a needle.

14. The ultrasound device according to claim 13, further comprising a disposable cap configured to be detachably coupled to the second end portion of the handle body for covering the at least one ultrasound transducer, wherein the cannula has a distal end portion configured to be detachably coupled to the cap.

15. The ultrasound device according to claim 1, wherein the display is slidable relative to the handle body.

16. An ultrasound device for guiding a needle, the ultrasound device comprising:
an elongated handle body having a first end portion and a second end portion;
a housing coupled to the second end portion of the handle body, the housing defining a channel configured for receipt of a needle; and
first and second ultrasound transducers disposed within the housing, wherein the first and second ultrasound transducers are disposed on opposite sides of the channel and angled toward a longitudinal axis defined by the channel.

17. The ultrasound device according to claim 16, wherein the first ultrasound transducer has a distally-oriented surface defining a first plane, and the second ultrasound transducer has a distally-oriented surface defining a second plane, the first and second planes disposed relative to one another at an angle of between 80 degrees and 170 degrees.

18. The ultrasound device according to claim 16, further comprising a coupling interface disposed within a cavity cooperatively defined by the first and second ultrasound transducers, the coupling interface fabricated from an acoustically-transparent material.

19. The ultrasound device according to claim 18, wherein the coupling interface defines a channel therethrough configured for passage of a needle, the channel of the coupling interface coaxial with the channel of the housing.

20. The ultrasound device according to claim 18, wherein the coupling interface has a planar, base surface and each of the first and second ultrasound transducers has a planar, base surface disposed at an acute angle relative to the base surface of the coupling interface.
